(19) United States
(12) Patent Application Publication
(10) Pub. No.: US 2021/0220379 A1
(43) Pub. Date: Jul. 22, 2021

(54) PERITONEAL SODIUM-GLUCOSE TRANSPORTER (SGLT) INHIBITORS FOR IMPROVEMENT OF PERITONEAL DIALYSIS

(71) Applicant: MOR RESEARCH APPLICATIONS LTD., Tel Aviv (IL)

(72) Inventors: Marina VOROBIOV, Beer Sheva (IL); Amos DOUVDEVANI, Beer Sheva (IL); Yosef HAVIV, Omer (IL); Boris ROGCHEV, Beer Sheva (IL)

(21) Appl. No.: 16/642,989

(22) PCT Filed: Aug. 30, 2018
(86) PCT No.: PCT/IB2018/056625

§ 371 Date: Feb. 28, 2020

Related U.S. Application Data
(60) Provisional application No. 62/551,805, filed on Aug. 30, 2017.

(51) Int. Cl. A61K 31/7034; A61M 1/28

(52) U.S. Cl. CPC A61K 31/7034; A61M 2210/1017; A61M 1/287

(57) ABSTRACT

Peritoneal dialysis fluid (PDF) comprising at least one inhibitor of glucose transport and compositions comprising same are provided. Use of a PDF and a glucose transport inhibitor such as a sodium-glucose co-transporter (SGLT) inhibitor decreases glucose absorption into the circulation of patients treated with peritoneal dialysis and, therefore, provides improved means for treating renal failure diseases.

### FIELD OF THE INVENTION

The present invention relates to treatment of kidney failure by using sodium glucose transporter inhibitors in peritoneal dialysis fluid.

### BACKGROUND

Peritoneal dialysis (PD) is one of the treatment modalities available for end stage kidney disease (ESKD). In this procedure, peritoneal dialysis fluid is introduced into the peritoneal cavity of a subject. The advantages of PD include: ease of access and training, better preservation of residual renal function, minimizing the need for hospitalization, wide range of regimens available for individualizing of prescription, minimal interference with life, physical and mental well-being and others.

Most frequently used commercial peritoneal dialysis fluids (PDFs) contain high glucose concentration, often in the 100 mM range, as the major osmotic agent. Glucose as an osmotic agent is safe, reasonably priced, easy to sterilize and relatively stable. Introduction of this relatively high molarity fluid into the peritoneal cavity causes both excessive fluid removal owing to high osmotic gradient, and osmotic extraction of various waste molecules (such as urea and creatinine), which are normally removed from the blood by the kidneys. However, during dwell of the PDF in the peritoneal cavity a considerable amount of glucose (about 60%-80%) is absorbed into the blood. Glucose blood absorption has two adverse consequences (a) it decreases the osmotic gradient during PD; and (b) blood glucose elevation causes undesirable metabolic consequences including hyperosmolar stress, hyperlipidemia, obesity, appetite and nutritional alterations. Even without the added glucose load, uremic patients have abnormal glucose and insulin metabolism with glucose intolerance, hyperinsulinemia, and insulin resistance. In diabetic patients, massive peritoneal glucose absorption may cause instability of blood glucose levels, increase in insulin dose requirement, and ultimately the development or progression of diabetic complications.

Active glucose transport is involved in glucose transport during PD, mainly mediated by a glucose transporter (GLUT) and sodium-glucose co-transporters (SGLTs). For example, presence of the SGLT isoform SGLT1 on the apical surface of peritoneal mesothelial cells and GLUT expression on the basolateral surface of these cells, suggests that these transporters mediate glucose uptake from the lumen, followed by transcellular transport of glucose (Schroppel et al., (1998) Kidney Int; 53(5):1278-87).

Little is known on the presence and function of other SGLT isoforms in the peritoneum.

Sodium-glucose co-transporters inhibition became a target for diabetes therapy and broadened the spectrum of glucose-lowering agents. For example, phlorizin a non-specific SGLT inhibitor, first isolated from that the bark of apple trees, has been long known for its ability to decrease glucose levels in the blood and increase insulin sensitivity. Phlorizin inhibits bot the SGLT1 isoform that is involved in intestinal glucose uptake and SGLT2 that is involved in kidney glucose reuptake, but it has poor oral bioavailability and it causes several gastrointestinal side effects. Phlorizin-based analogs with improved bioavailability and stability, as well as selectivity such as dapagliflozin, canagliflozin, and empagliflozin (SGLT2 inhibitors) are now available and approved for use in humans.

There is an unmet medical need for compositions and methods of treating patients with kidney failure using peritoneal dialysis fluids that will decrease or minimize the absorption of glucose into the circulation of the patients and thus eliminate complications associated with PD.

### SUMMARY

The present disclosure provides the combined use of a peritoneal dialysis fluid (PDF) and one or more inhibitors of sodium-glucose co-transporters (SGLT inhibitors) as a means to obtain an improved peritoneal dialysis (PD) treatment for end stage kidney disease (ESKD) patients. The improvement resides inter alia in significantly reducing or minimizing absorption of glucose, the main osmolyte in the PDF, from the peritoneal cavity to the blood stream. By providing means to at least partially block glucose absorption, at least two aspects of conventional PD are dramatically improved: (a) the osmotic gradient during PD is maintained; and (b) blood glucose elevation during dialysis is restrained or arrested, thereby reducing and even eliminating common undesirable metabolic consequences associated with elevated blood glucose level such as hyperosmolar stress, hyperlipidemia, obesity, appetite and nutritional alterations. Such improvements are particularly valuable to diabetes patients treated with peritoneal dialysis.

In one aspect, the present disclosure provides a PDF comprising at least one SGLT inhibitor. For example, a disclosed PDF may comprise a single SGLT inhibitor, two SGLT inhibitors or even more.

In another aspect, the present disclosure provides a pharmaceutical composition comprising a dialysis fluid, at least one SGLT inhibitor, and a pharmaceutically acceptable excipient. The dialysis fluid may be, for example a PDF.

A disclosed PDF and/or a disclosed pharmaceutical composition may be used in the treatment of a kidney failure disease disorder or condition.

In a further aspect of the present disclosure, methods of treatments are provided, comprising administering to a subject in need thereof an effective amount of a PDF of the present disclosure comprising one or more SGLT inhibitors or a pharmaceutical composition comprising same, and/or administering to the subject effective amounts of a PDF and of one or more SGLT inhibitors, thereby treating the subject.

Methods provided herein are suitable, for example, for treatment of a subject inflicted with a kidney failure disease, disorder or condition; for reducing glucose absorption into the blood of a subject undergoing peritoneal dialysis; and/or for treating a subject in need of a peritoneal dialysis.

A disclosed method may afford a reduction in peritoneal membrane damage as well as reduced blood glucose levels in a subject undergoing PD.

A PDF of the disclosure comprising one or more SGLT inhibitors may be administered into the peritoneal cavity of the subject. This form of administration of a SGLT inhibitor can circumvent several gastrointestinal side effects such as diarrhea, dehydration and malabsorption often associated with oral administration of some SGLT inhibitors and, in addition, improve bioavailability of some inhibitors such as phlorizin.

A PDF and at least one SGLT inhibitor may be designed and administered as a single unit dosage form. Alternatively or additionally, a PDF and at least one SGLT inhibitor may be designed and administered as two or more unit dosage forms, wherein PDF is intraperitoneally administered and one or more SGLT inhibitors may be administers in the same or different route, for example, a SGLT inhibitor may be administered orally and/or subcutaneously.

In a further aspect, the present disclosure provides a kit comprising a PDF and one or more SGLT inhibitors and, optionally, instructions and means for applying the kit to a subject in need thereof. For example, a disclosed kit is a peritoneal dialysis kit, useful e.g., for treatment of a renal failure disease, disorder or condition.

At least one of the SGLT inhibitors, in any one of the aspects of the disclosure, is a SGLT1 inhibitor, a SGLT5 inhibitor, a dual SGLT1/SGLT5 inhibitor or a SGLT inhibitor capable of inhibiting SLGT-2 and at least one of SGLT1 and SGLT5. When at least two SGLT inhibitors are applied, they comprise a SGLT1 inhibitor and a SGLT5 inhibitor.

In some embodiments, the SGLT inhibitor is selected from phlorizin, a phlorizin analog, a phlorizin derivative and any combination thereof. For example, a phlorizin analog may be selected from an O-glucoside analog or a C-glucoside analog. In exemplary embodiments, the SGLT inhibitor is capable of inhibiting SGLT1. In certain embodiments, the SGLT inhibitor is selective to SGLT1. In exemplary embodiments, the SGLT inhibitor is capable of inhibiting SGLT1 and SGLT2 and/or be selective to SGLT5.

The amount of one or more SGLT inhibitors in a disclosed PDF, pharmaceutical composition, kit and/or method may be from about 0.1 to about 50 μM or from about 0.2 to about 20 μM.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a photograph of 2% agarose gel runs stained with ethidium bromide, depicting the presence SGLT1 and SGLT15 DNA isolated from mesenteric peritoneum, kidney and small intestine of CD-1® mice;

FIG. 2 is a graph showing glycosuria score measured by standard urine stick following single subcutaneous (SC) phlorizin injection to healthy CD-1® mice (n=6);

FIGS. 3A-3C are graphs showing glucose concentrations measured in blood (3A) and peritoneal fluid (3C) of CD-1® mice induced with end stage renal failure and, 24 hour later, injected SC with saline (Control) or phlorizin and exposed to peritoneal dialysis (n=5 in each group). FIG. 3B presents area under the curve (AUC) of blood glucose levels obtained from all mice. Glucose levels in peritoneal fluid were measured at 30 min. *P<0.05;

FIG. 4 is a graph showing blood glucose concentration in CD-1® mice (n=5) with end stage renal failure, subcutaneously injected with either phlorizin or saline (control), measured after intraperitoneal injection of sodium-free PDF;

FIGS. 5A-5C are graph showing blood glucose concentrations measured in CD-1® mice induced with end stage renal failure, and 24 hour later intra-peritoneally injected with phlorizin-free PDF (5A; Control) or with PDF containing 0.6 μM phlorizin (5B). Four mice were studied in each group, represented by separate curves in the graphs. FIG. 5C presents area under the curve (AUC) of blood glucose levels obtained from all mice (P<0.01);

FIGS. 6A-6B are graph showing blood glucose concentration measured in male rats induced with end stage renal failure, and 24 hour later provided with a temporary catheter introduced into their peritoneal cavity. Rats were infused either with phlorizin-free, PDF (Dianeal®) or with PDF (Dianeal®) supplemented with phlorizin (6A). FIG. 6B presents AUC of blood glucose levels obtained from all mice (P<0.01);

FIGS. 7A-7B are a graph (7A) and bar graph (7B) showing glucose concentration in peritoneal dialysis fluid (PDF) and AUC, respectively, measured in the same rats treated as described in FIG. 6A-6B (p<0.001); and

FIGS. 8A-8B are a graph (8A) and bar graph (8B) showing FITC-dextran concentration in peritoneal dialysis fluid (PDF) and AUC, respectively, measured in the same rats treated as described in FIG. 6A-6B (P<0.05).

### DETAILED DESCRIPTION

The present disclosure relates, at least in part, to the application of sodium glucose transporter inhibitors in peritoneal dialysis in treatment of kidney failure.

The present disclosure is based on a discovery by the present inventors that glucose absorption from the peritoneum into the blood circulation, in the course of a peritoneal dialysis, may be substantially lowered by inhibiting certain glucose pumps or transporters residing in the peritoneum. Clearly, inhibition of peritoneal glucose absorption, has a potentially important positive impact in prevention of numerous dialysis-related complications, and utilization of peritoneal dialysis (PD) modality in a global context.

The present inventors have established animal models showing the advantageous effects of inhibiting active trans-cellular peritoneal glucose transporters such as sodium-glucose co-transporters (SGLTs) in decreasing glucose transport into the systemic circulation during peritoneal dialysis. Disclosed herein, for the first time, is experimental evidence, using renal failure mouse and rat models, of the role of SGLT in glucose absorption from the peritoneal cavity. As exemplified herein below, animals exposed to intraperitoneal dialysis fluid following administration of the SGLT blocker phlorizin showed significantly lower blood glucose levels, and a higher residual glucose concentration in the peritoneal dialysis fluid (PDF), suggesting slower and/or lower glucose absorption from the peritoneal cavity. Prevention or reduction of transcellular peritoneal glucose transport via inhibition of SGLT can provide an improved PD procedure. Indeed, these findings provide the basis for utilizing glucose transporter inhibitors for improvement of peritoneal dialysis fluids and thereby improving the clinical outcome of PD.

Six SGLT isoforms have been described so far (Van Steenbergen et al. 2017, Sci Rep. 27(7):41166; Grempler et al. 2012, FEBS Lett. 586(3):248-53). SGLT1 is mainly expressed in the intestines, particularly in the small intestine and in the kidneys. It is responsible for active glucose absorption in both tissues. SGLT2 is mainly expressed in the kidneys, playing a major role in glucose reabsorption in tubules. SGLT3 has been detected mainly in the enteric nervous system of the intestine and in skeletal muscle and is hypothesized to be a glucose-sensor. Compared to SGLT1 and SGLT2, its affinity for glucose is rather low and the binding of sugar to human SGLT3 triggers membrane depolarization without any sugar transport. SGLT4 has been characterized as a sodium-dependent mannose and fructose transporter in the small intestine and in the kidney.

Sodium-glucose co-transporter-5 (SGLT5) was previously reported to be expressed exclusively in the kidneys where it reabsorbs glucose and galactose, but has been also shown to be expressed in, e.g., liver, bovine testes, porcine jejunum, skeletal muscle and spleen. SGLT6 is expressed in the brain and kidneys and its affinity for glucose is rather low.

The expression of several sugar transporter isoforms in individual tissues and cells is a reflection of the different characteristics of each of the various transporters and provides a high degree of specificity in the control of glucose uptake under different physiological conditions. The different SGLT isoforms transport glucose with different affinities, via a secondary active transport mechanism. This form of glucose transport takes place across the luminal membrane of cells lining the small intestine and the proximal tubules of the kidneys.

The expression profile of SGLT isoforms SGLT1 and SLGT5 in the peritoneal membrane was further verified by the present inventors. The present disclosure expands the knowledge of SGLTs functions in renal failure and their role in glucose absorption during PD treatment. As such, it is now disclosed, for the first time, that SGLT1 and SGLT5 are present in the peritoneal membranes. Based on this discovery, the present inventors envisaged inhibition of these SGLTs subtypes as a valuable and useful means to prevent or reduce glucose absorption from PDF in peritoneal dialysis patients.

### Peritoneal Dialysis Fluids, SGLT Inhibitors and Formulations Thereof

In an aspect of the present disclosure, there is provided a peritoneal dialysis fluid comprising one or more SGLT inhibitors.

Dialysis is a way of cleaning the blood when the kidneys can no longer do this function. As the kidneys lose their ability to function, fluid, minerals, and waste products that are normally removed from the body in the urine begin to build up in the blood. When these problems reach a critical stage, excess fluid and waste must be removed either by getting a kidney transplant or with kidney (renal) replacement therapy, namely, dialysis. There are two kinds of dialysis. In hemodialysis, blood is pumped out of the body to an artificial kidney machine and returned to the body by tubes that connect to the machine. In peritoneal dialysis, the peritoneum, namely, the serous membrane lining the abdominal cavity acts as a natural filter. Wastes are taken out by means of a cleansing fluid called dialysate or dialysis fluid, which is infused or washed in and out of the peritoneal cavity cavity in cycles. The fluid is held within the abdomen for a prescribed period of time called “a dwell”. When the dwell is completed, the “used” dialysate containing the excess fluid and waste that has been removed from the blood (normally eliminated in the urine) can then be drained out of the abdomen e.g., into a sterile container. The peritoneal cavity is then filled again with fresh dialysate, and the process starts again.

The term “peritoneal dialysis fluid” (PDF), as used herein, refers to the fluid that is used in the type of dialysis that uses the peritoneum of patients as the membrane through which fluid and dissolved substances are exchanged with the blood. The components of PDF include, but are not limited to electrolytes, buffer and osmotic agents. For example, a standard PDF typically contains from about 0.55% to about 4.25%, for example, 1.5%, 2.5%, or 4.25%, by weight glucose or dextrose, sodium, calcium, magnesium, chloride and lactate. Any PDF that is used for peritoneal dialysis may be used in any one of the aspects and embodiments of the present disclosure.

Standard peritoneal dialysis fluid contains varying concentrations of glucose, in the form of dextrose, as the osmotic agent. Therefore, the dialysate is hyperosmolar in relation to serum, causing fluid efflux (ultrafiltration) to occur. The volume of ultrafiltration depends on the concentration of glucose solution used for each exchange, the length of time the fluid dwells in the peritoneal cavity, and the individual patient's peritoneal membrane characteristics. With increasing dwell time, transperitoneal glucose absorption diminishes the dialysate glucose concentration and the osmotic gradient.

The standard dialysate contains calcium in concentrations ranging from 1.25 to 1.75 mmol/L. Such concentrations of calcium typically result in the movement of calcium from the PD solution to the extracellular fluid, potentially helping to support blood pressure in the critically ill patient. Standard dialysate does not contain potassium, but it contains sodium (132 mEq/L/mmol/L) and magnesium (0.5-1.5 mEq/L/0./25-0.75 mmol/L). Other agents, such as heparin, insulin, antibiotics, and potassium, may be added to the dialysate as the clinical situation dictates.

The commonly-used commercially available PD fluid Dianeal®, for example, Dianeal PD-2, Dianeal PD-4 and 1 mmol/L Calcium peritoneal dialysis solution, contain the electrolytes sodium, chloride, calcium, and magnesium, dextrose (D-glucose) as an osmolyte, and lactate as a buffer. Absorbed lactate from PD fluid is converted to bicarbonate in the body, allowing a lactate gradient to be maintained. The lactate in PD fluid also regulates the formation of glucose degradation products (GDPs). Peritoneal dialysis fluid also contains NaCl.

Another approach to PD is through the simultaneous use of glucose-based and amino acid-based dialysates that are mixed immediately before administration with an automated device. The absorption of amino acids during a dwell is higher than that of glucose because of their lower molecular weight. This approach has the theoretic advantage of reducing amino acid loss and improving nitrogen balance, but their use is limited because of the nitrogen load.

The use of SGLT inhibitors may advantageously inhibit or minimize the absorption of glucose form the PDF into the systemic circulation of a dialysis patient, thereby affording a PDF which will minimally alter blood glucose levels of peritoneal dialysis patients and be of a highest value particularly, but not exclusively, to diabetic patients undergoing peritoneal dialysis. Hence, peritoneal dialysis treatment in kidney failure in general and of Type 2 diabetes patients, in particular, may benefit from the use of a contemplated PDF.

The SGLT inhibitors, may be, for example, natural phlorizin, a synthetic analog and/or a derivative thereof, wherein phlorizin is a molecule having the chemical name phloretin-2′-β-D-glucopyranoside, and the chemical structure:

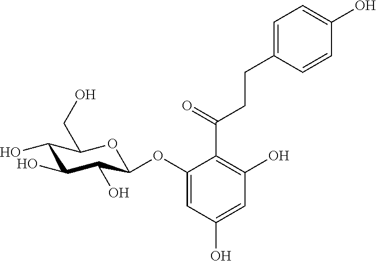

Phlorizin is a glucoside (a glycoside derived from glucose) belonging to a family of bicyclic flavonoids.

Phlorizin, according to embodiments described herein, may be derived, isolated or extracted from natural sources, such as tree bark, for example, apple, pear, or cherry bark, e.g., from root or trunk bark, or from other parts of these as well as other trees and plants. Additionally or alternatively, phlorizin may be produced synthetically.

The term “phlorizin derivative” refers herein to a phlorizin synthetic analog with one or more structural modifications such as, but not limited to, replacement of the natural/original moiety bonded to the glucose group with other moieties such as aromatic aldehydic or phenolic moieties, and/or conversion or replacement of one or more hydroxyl (—OH) groups to other functional groups, for example, esters or carboxylic groups. Drug development has focused on modifying the chemical structure of the natural SGLT inhibitor phlorizin and its synthetic analogs in order to control blood glucose concentrations in Type 2 diabetes patients and certain complications of the disease, including dialysis-related complications, and some SGLT2-specific phlorizin derivatives or analogs have been approved for treatment of Type 2 diabetes patients.

Phlorizin derivatives include, but are not limited to, O-glucoside analogs, namely analogs in which the glucose group is bonded to an anomeric moiety via an oxygen atom, and C-glucoside analogs in which the glucose group is bonded directly to an aromatic carbon of a covalently-linked moiety. Non-limiting examples of O-glucoside analogs include T-1095, sergliflozin, remogliflozin and AVE2268. Non-limiting examples of C-glucoside analogs include Sapagliflozin (Farxiga®), canagliflozin (Invokana®) and empagliflozin (Jardiance®).

The O-glucoside analogs of phlorizin have improved bioavailability and stability features. For example, T-1095 is absorbed into the circulation via oral administration, then metabolized to the active form, T-1095A, and suppresses the activity of SGLTs in the kidney.

Although known O-glucoside inhibitors show minimized glucosidase-mediated degradation and enhanced systemic exposure, they still have poor pharmacokinetic stability and incomplete pharmacological selectivity for SGLT2. The C-glucoside analogs, particularly dapagliflozin, canagliflozin and empagliflozin, were thus synthesized in order to have higher potency for SGLT2 versus SGLT1 and were approved as a medication for the treatment of Type 2 diabetes. These and other C-glucoside analogs of phlorizin which can be employed in embodiments described herein are disclosed, for example, in International Application Publication No. WO 2015128853, U.S. Pat. Nos. 7,851,502, and 8,221,786 (dapagliflozin); U.S. Pat. Nos. 7,943,582, 7,943,788, 8,222,219 and 8,513,202 (canagliflozin); and U.S. Pat. Nos. 7,579,449, 7,713,938 and 8,551,957 (empagliflozin). Numerous examples of further synthetic phlorizin analogs or derivatives that are specific inhibitors of SGLT2 are disclosed, for example, in U.S. Pat. Nos. 9,562,029 and 8,603,989 and U.S Appication Publication No. 2005/209166.

Peritoneal dialysis fluids comprising phlorizin, or a phlorizin derivative or analog are referred to herein as “phlorizin peritoneal dialysis fluid” (PPDF).

Further SGLT blockers include, for example, drugs developed for the treatment of Type 1 and Type 2 diabetes mellitus. Non-limiting examples of such drugs include sotagliflozin, ipragliflozin and mizagliflozin. Sotagliflozin or LX4211, was developed for oral delivery and inhibits both SGLT2 and SGLT1. Ipragliflozin is a SGLT2 inhibitor developed for treatment of diabetes and diabetic nephropathy. Mizagliflozin is a SGLT1 inhibitor developed for treatment of constipation. Additional SGLT blockers, for example SGLT1 and SGLT2 inhibitors are disclosed, for example, in Choi, 2016, Molecules, 21:1-12.

Any SGLT blockers currently available or to be developed or produced in the future, which inhibit and/or selectively inhibit one or more of the SGLT isoforms present in the peritoneum, are contemplated herein.

In some embodiments of any aspect of the disclosure, the SGLT inhibitors are specific inhibitors of SGLT1 and/or SGLT5. For example, the SGLT inhibitor is either an SGLT1 inhibitor or an SGLT5 inhibitor. In some embodiments, the SGLT inhibitor is a dual SGLT1/SGLT5 inhibitor.

In some embodiments, the SGLT inhibitor is capable of inhibiting at least one of SGLT1 and SGLT5, present in the peritoneal membrane, and is also capable of binding to SGLT2.

Some embodiments described herein concern the use of phlorizin, a phlorizin analog and/or a phlorizin derivative as one or more SGLT blockers or inhibitors. For example, the SGLT inhibitor may be a phlorizin derivative capable of inhibiting SGLT1. In exemplary embodiments, the SGLT inhibitor is selective to SGLT1. The phlorizin derivative used for inhibiting SGLT1 may be, for example, an O-glucoside analog such as, but not limited to, T-1095, remogliflozin or sergliflozin. The phlorizin derivative used for inhibiting SGLT1 may alternatively or additionally be a C-glucoside analog such as, but not limited to, dapagliflozin, canagliflozin or empagliflozin.

The SGLT inhibitor may be, in some embodiments, phlorizin, a synthetic analog and/or derivative thereof capable of inhibiting SGLT5. In exemplary embodiments, the SGLT phlorizin inhibitor is selective to SGLT5.

In some embodiments, the SGLT inhibitor is a dual SGLT1/SGLT2 inhibitor. In exemplary embodiments the dual SGTL1/SGLT2 inhibitor is Sotagliflozin.

Inhibitors or blockers of SGLTs have been used in the art mainly for treatment of diabetic patients. The use of SGLT inhibitors in dialysis, particularly peritoneal dialysis is disclosed herein for the first time.

A SGLT inhibitor may be provided to a patient during dialysis as a distinct or separate dosage forms administered either before or concomitantly with administering a dialysis fluid, for example a PDF. Alternatively or additionally, a SGLT and a dialysis fluid may be combined together and formulated as single unit dosage form for dialysis.

Contemplated by the present disclosure are peritoneal dialysis fluids comprising one or more SGLT inhibitors. To this end, any of the known DF may be useful for the preparation of a PDF containing one or more SGLT inhibitors. Non-limiting examples include, Dianeal® 1.5%, 2.5% and 4.25% dextrose.

A SGLT inhibitor that is given directly in the peritoneal dialysis fluid so as to decrease glucose reabsorption by specifically targeting SGLTs, presents an advanced approach in peritoneal dialysis. Such improved PDFs used in treatment of end stage kidney disease patients may provide reduced glucose load during PD, improved patients' outcome and less peritoneal membrane damage.

A desired concentration of a SGLT may be determined based on the vast knowledge in the art pertaining, for example, to use of SGLTs in treatment of Type I and/or Type II diabetes. For example, Hummel et al. 2012 (Am J Physiol Cell Physiol. 15; 302 (2):C373-382) examined in vitro the compounds dapagliflozin and phlorizin, and tested their kinetics of interaction with human SGLT1 (hSGLT1) and human SGLT2 (hSGLT2) as the basis of selectivity for hSGLT2 over hSGLT1. The IC50 of phlorizin to SGLT1 was 0.4 μM in the intestines. In the kidney, the IC50 of phlorizin for inhibition of glucose re-uptake was 0.065 μm.

In some embodiments a SGLT inhibitor is applied in a concentration range of from about 0.1 μM to about 50.0 μM. For example, from about 0.1 μM to about 0.5 μM, from about 0.3 μM to about 0.8 μM, from about 0.5 μM to about 1.0 μM, from about 0.8 μM to about 10.0 μM, from about 1.5 μM to about 5.0 μM, from about 3.5 μM to about 10.0 μM, from about 8.5 μM to about 15.5 μM, from about 10.0 μM to about 20.0 μM, from about 10.5 μM to about 15.0 μM, from about 13.0 μM to about 25.0 μM, from about 20.0 μM to about 30.0 μM, from about 25.5 μM to about 45.0 μM, or from about 40.0 μM to about 50.0 μM, of a SGLT inhibitor, for example, a SGLT1 and/or SGLT5 inhibitor.

For example, the concentration of a SGLT inhibitor used in any one of the aspects and embodiments of the disclosure may be from about 0.2 to about 20 μM, or from about 0.4 to about 10 μM, for example, about 0.6 μM.

A disclosed PDF may comprise one or more SGLT inhibitors. In some embodiments, a contemplated PDF comprises a single SGLT inhibitor. For example, a SGLT1, SGLT5, SGLT2 or combined SGLT1/SGLT2 inhibitor as defined herein.

In some embodiments, a contemplated PDF comprise at least two different SGLT inhibitors, for example SGLT1 inhibitor and a SGLT5 inhibitor as defined herein.

In a further aspect, the present disclosure provides a composition comprising a peritoneal dialysis fluid, one or more SGLT inhibitors and a pharmaceutically acceptable excipient. In some embodiments, the composition is a formulation for pharmaceutical administration, and comprises a pharmaceutically acceptable carrier. A contemplated formulation may be used, for example, for treatment of a subject inflicted with kidney failure and is in need of dialysis, particularly when it is necessary to decrease or minimize glucose absorption during dialysis and a consequent blood glucose level elevation.

The PDF and the one of more SGLT inhibitors are as described in any one of the embodiments disclosed herein. For example, in some embodiments, the SGLT inhibitor in accordance with a contemplated formulation is a SGLT1 inhibitor, for example, but not limited to, a SGLT inhibitor which is selective to SGLT1 such as an O-glucoside analog or a C-glucoside analog of phlorizin (e.g., T-1095, remogliflozin and sergliflozin, apagliflozin, canagliflozin, and empagliflozin).

In some embodiments, the SGLT inhibitor is SGLT5, for example, a SGLT inhibitor which is selective to SGLT5. In some embodiments, the SGLT inhibitor is a dual SGLT1 and SGLT5 inhibitor. In some embodiments, the SGLT inhibitor is a dual SGLT1/SGLT2 inhibitor. In some embodiments, the SGLT inhibitor is capable of inhibiting SGLT2 and at least one of SGLT1 and SGLT5.

In some embodiments, a contemplated pharmaceutical composition comprises one SGLT inhibitor, for example a SGLT1 inhibitor or a dual SGLT1 and SGLT5 inhibitor.

In some embodiments, a contemplated pharmaceutical composition comprises at least two SGLT inhibitors, for example a SGLT1 inhibitor and a SGLT5 inhibitor.

The term “pharmaceutical composition”, as used herein, refers to a formulation designed for medicinal utilization such as, but not limited to, therapeutic or diagnostic utilization. “Formulation” as used herein refers to any mixture of different components or ingredients prepared in a certain way, i.e., according to a particular formula. For example, a formulation may include one or more active pharmaceutical ingredients (APIs) combined or formulated together with, for example, one or more carriers, excipients, stabilizers and the like. The formulation may comprise solid and/or non-solid, e.g., liquid, gel, semi-solid (e.g. gel, wax) or gas components. Usually, in a formulation for pharmaceutical administration the APIs are combined or formulated together with one or more pharmaceutically and physiologically acceptable carriers, which can be administered to a subject (e.g., human or non-human subject) in a specific form, such as, but not limited to, tablets, linctus, ointment, infusion or injection. A pharmaceutical composition is interchangeably used herein with term “formulation” with reference to medicinal formulation.

Some embodiments described herein pertain to liquid pharmaceutical compositions, for example aqueous formulations, comprising a PDF and at least one SGLT inhibitor.

In some embodiments, a contemplated pharmaceutical composition, e.g., formulation, is a suspension.

The terms “active agent”, “active ingredient” and “active pharmaceutical ingredient (API)” as used herein are interchangeable, all of which refer to a compound, which is accountable for a desired biological or chemical effect, for example, inhibition of a sodium-glucose co-transport. In the context of embodiments described in the present disclosure, the terms API or active agent also encompasses a peritoneal dialysis fluid.

As used herein, the terms “pharmaceutically acceptable”, “pharmacologically acceptable” and “physiologically acceptable” are interchangeable and mean approved by a regulatory agency of the Federal or a state government or listed in the U.S. Pharmacopeia or other generally recognized pharmacopeia for use in animals, and more particularly in humans. These terms include formulations, molecular entities, and compositions that do not produce an adverse, allergic or other untoward reaction when administered to an animal, or a human, as appropriate. For human administration, preparations should meet sterility, pyrogenicity, general safety and purity standards as required by, e.g., the U.S. Food and Drug Administration (FDA) agency, and the European Medicines Agency (EMA).

A contemplated pharmaceutical composition may, optionally, further comprise one or more physiologically acceptable excipients and/or a physiologically acceptable carrier.

Herein the term “excipient” refers to an inert substance added to a pharmaceutical composition (formulation) to further facilitate process and administration of the active ingredients. “Pharmaceutically acceptable excipients”, as used herein, encompass preservatives, antioxidants, coatings, isotonic and absorption delaying agents, and the like, that are compatible with pharmaceutical administration. Pharmaceutically acceptable excipients, as used herein, also encompass pharmaceutically acceptable carriers, namely, approved carriers or diluents that do not cause significant irritation to an organism and do not abrogate the biological activity and properties of a possible active agent. Physiologically suitable carriers in liquid medicinal formulations may be, for example, solvents or dispersion media. The use of such media and agents in combination with pharmaceutically active agents is well known in the art.

Excipients suitable for formulations described herein may comprise, for example, an enhancer (e.g., pyrrolidones, polyols, terpenes and the like) and/or a gelation agent (e.g., cellulose polymers, carbomer polymers and derivatives thereof), and/or a thickening agent (e.g., polysaccharides (agarose), polyacrylic polymers and the like).

In some embodiments, a disclosed pharmaceutical composition may further comprise one or more active agents, herein termed “secondary active agents” which may be added to the formulation so as to support, enhance, intensify, promote or strengthen the biological activity of the main or prime active agent(s). Additionally or alternatively, the secondary active compounds may provide supplemental or additional therapeutic functions. Non-limiting example of such additional active agents include inhibitors of other glucose transporters such as a GLUT inhibitor, antibiotics, pain killers and the like.

When a contemplated medicinal formulation comprises a PDF and one or more SGLT inhibitors, these APIs can be combined and formulated in the same formulation, namely, as a single unit dosage from or, alternatively, can be formulated in separate formulations, namely a plurality of dosage unit forms, for example, two or more dosage unit forms, each comprising one or more of a first active agent (e.g., a SGLT inhibitor), and/or a second active agent (i.e., a PDF).

A disclosed pharmaceutical composition may often comprise one or more antioxidants, namely, substances which slow down the damage that can be caused to other substances by the effects of oxygen (i.e., oxidation). Non-limiting examples of antioxidants include ascorbic acid (vitamin C) or a salt thereof (e.g., sodium ascorbate, calcium ascorbate, potassium ascorbate, ascorbyl palmitate, and ascorbyl stearate); cysteine or a cysteine derivative such as L-cysteine, N-acetyl cysteine (NAC), glutathione, a thiol precursor such as L-2-oxo-4-thiazolidine carboxylic acid (OTC), or a salt thereof; lipoic acid; uric acid; carotenes; α-tocopherol (vitamin E); and ubiquinol (coenzyme Q).

Further antioxidants are exemplified by phenolic antioxidants such as di-tert-butyl methyl phenols, tert-butyl-methoxyphenols, butylated hydroxyanisole (BHA), butylated hydroxytoluene (BHT), polyphenols, tocopherols, ubiquinones (e.g., caffeic acid, tert-butylhydroquinone (TBHQ)), propyl gallate, flavonoid compounds, cinnamic acid derivatives, coumarins, and sulfite salts such as sodium hydrogen sulfite or sodium bisulfite (e.g. sodium metabisulfite).

Contemplated formulations may contain a surfactant. Non-limiting examples of surfactants include polysorbate 20, 40, 60 and/or 80, (Tween®-20, Tween®-40, Tween®-60 and Tween®-80, respectively), Span 20, Span 40, Span 60, Span 80, Span 85, polyoxyl 35 castor oil (Cremophor EL), polyoxyethylene-660-hydroxystearate (macrogol 660), triton or Poloxamer 188 (Pluronic® F-68).

A contemplated pharmaceutical composition, e.g., medicinal formulation may comprise a buffer. Examples of buffers that may be used in accordance with described embodiments include, without limiting, citrate buffer, acetate buffer, sodium acetate buffer, tartrate buffer, phosphate buffer, borate buffer, carbonate buffer succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, or a mixture thereof.

A disclosed pharmaceutical composition may be formulated as a liquid, gel, cream, solid, film, emulsion, suspension, solution, lyophylisate or aerosol. Usually, a contemplated pharmaceutical composition comprising PDF is formulated as a liquid. When the pharmaceutical composition comprises a plurality of dosage unit forms, for example two dosage unit forms, these dosage unit forms can be formulated in different forms. For example, a first unit dosage form comprising, e.g. a PDF may be formulated as a liquid formulation, and the second unit dosage form comprising, e.g., a SGLT inhibitor such as phlorizin, an analog thereof or a derivative thereof, can be formulated as a solid formulation.

Disclosed pharmaceutical compositions may be formulated for any suitable route of administration, e.g., for subcutaneous, transdermal, intradermal, transmucosal, intravenous, intraarterial, intramuscular, intraperitoneal, intratracheal, intrathecal, intraduodenal, intrapleural, intranasal, sublingual, buccal, intestinal, intraduodenally, rectal, intraocular, or oral administration. The compositions may also be formulated for inhalation, or for direct absorption through mucous membrane tissues.

In embodiments described herein, the pharmaceutical compositions disclosed are aqueous formulations particularly useful for intraperitoneal administration e.g., via a catheter.

Unit dosage form, in a contemplated formulation, that comprise at least one SGLT inhibitor, may be designed for oral or buccal administration, and may be formulated as tablets, pills, dragees, capsules, liquids, gels, syrups, slurries, suspensions, and the like well Such compositions may further comprise one or more excipients selected from sweetening agents, flavoring agents, coloring agents and preserving agents in order to provide pharmaceutically elegant and palatable preparations. A unit dosage form that comprise at least one SGLT inhibitor may designed for administration by inhalation and delivery, e.g., as an aerosol spray. Alternatively, the unit dosage form may be designed for rectal administration as suppositories or retention enemas. Contemplated pharmaceutical compositions may also comprise one- or more unit dosage forms comprising one or more SGLT inhibitors formulated for local administration, such as subcutaneously or intramuscularly administration, intramuscular injection or topical administration.

In some embodiments, a contemplated formulation is designed for parenteral administration, e.g., by bolus injection or continuous infusion. Injectable formulations may be suspensions, solutions, e.g., aqueous solutions, or emulsions in oily or aqueous vehicles, and may contain excipients such as suspending, stabilizing, dispersing agents, substances which increase the viscosity of a suspension, and/or agents which increase the solubility of the compounds to allow for the preparation of highly concentrated solutions. Alternatively, the active ingredient(s) may be in powder form for constitution with a suitable vehicle, e.g., sterile, pyrogen-free water, before use.

All compositions for any form of administration may be prepared according to any method known in the art for the manufacture of pharmaceutical compositions.

### Methods of Treatment

In an aspect of the disclosure, there is provided a method of treatment of a subject inflicted with a kidney failure disease, disorder or condition, comprising administering to the subject an effective amount of a dialysis fluid and one or more SGLT inhibitors, thereby threating the subject.

In some embodiments, the dialysis fluid is a peritoneal dialysis fluid (PDF) as defined herein, being administered into the peritoneal cavity of the subject.

Kidney failure, also termed end-stage renal disease (ESRD), is the last stage of chronic kidney disease. When the kidneys fail (i.e., lose the ability to filter waste and toxins from the blood sufficiently), it means they have stopped working well enough for a subject to survive without dialysis or a kidney transplant. The term “chronic kidney disease” means lasting damage to the kidneys that can get worse over time. If the damage is very bad, the kidneys may stop working (namely, ESRD). Chronic kidney disease (CKD) usually gets worse slowly, and symptoms may not appear until the kidneys are badly damaged. In the late stages of CKD, while nearing kidney failure, a subject may notice symptoms that are caused by waste and extra fluid building up in the body (e.g., itching, muscle cramps, nausea and vomiting, swelling of feet and ankles, too much urine or not too much urine, unexplained shortness of breath and excessive drowsiness or fatigue).

In most cases, kidney failure is a complication of, caused by, or is a sequela of, other health problems that have caused permanent damage (harm) to the kidneys little by little, over time. Diabetes is the most common cause of ESRD. High blood pressure is the second most common cause of ESRD. Other problems that can cause kidney failure include, for example, autoimmune diseases, such as lupus and IgA nephropathy, genetic diseases such as polycystic kidney disease, nephrotic syndrome, urinary tract problems and toxic exposure to environmental pollutants or certain medication.

In some embodiments, kidney failure may be a disorder or condition selected from a comorbidity disease, a side effect, and/or a complication of another disease or disorder. In some embodiments, kidney failure is a secondary disease.

Sometimes the kidneys can stop working very suddenly (within two days). This type of kidney failure is a condition or disorder called acute kidney injury or acute renal failure. Common causes of acute renal failure include for example, kidney trauma e.g., as result of accident, heart attack, severe dehydration, illegal drug use and drug abuse, not enough blood flowing to the kidneys and urinary tract problems. This type of kidney failure is not always permanent, and the kidneys may go back to normal or almost normal with treatment and avoidance of other serious health problems.

The terms “kidney failure disease, disorder or condition” and “renal failure disease, disorder or condition” are interchangeable, and used herein so as to encompass all categories and definitions of kidney/renal failure or ESRD as defined herein and/or known in the art, for example, a CKD, a complication, side effect, secondary disease or disorder or a sequela of other health problems such as those defined above, a comorbidity, and acute kidney injury.

In some embodiments, a subject inflicted with a kidney failure disease, disorder or condition is in need of a peritoneal dialysis. The present disclosure therefore provides, in accordance with these embodiments, a method of treating a renal failure disease, disorder or condition in a subject, comprising at least the following steps:

administering an effective amount of a peritoneal dialysis fluid (PDF) into the peritoneal cavity of the subject; and

administering an effective amount of at least one SGLT inhibitor to the subject, thereby treating renal failure disease, disorder or condition in the subject.

In a further aspect, the present disclosure provides a method of reducing glucose absorption into the blood of a patient undergoing peritoneal dialysis, the method comprising the steps of:

administering an effective amount of a peritoneal dialysis fluid into the peritoneal cavity of the subject; and

administering an effective amount of at least one SGLT inhibitor to the subject, thereby reducing the absorption of glucose into the circulation of the patient during peritoneal dialysis.

In yet a further aspect, the present disclosure provides a method of treating a subject in need of a peritoneal dialysis with at least one SGLT inhibitors during peritoneal dialysis. The method of the present disclosure may be applied in conjunction with any method known in the art for treatment of dialysis patients, including ESKD patients and diabetic patients. Treatment with one or more SGLT inhibitors may be reasoned or may be needed in order to reduce the absorption of glucose into the subject's circulation. In some embodiments a method of treating a subject in need thereof a peritoneal dialysis, comprises the steps of:

introducing PDF into the peritoneal cavity of the subject; and

introducing at least one SGLT inhibitor, optionally, into the peritoneal cavity of the subject, thereby treating the subject in need of a peritoneal dialysis while reducing the absorption of glucose into the subject's circulation.

The peritoneum is a continuous membrane which lines the abdominal cavity and covers the abdominal organs (abdominal viscera). It acts to support the viscera and provides pathways for blood vessels and lymph to travel to and from the viscera. The peritoneum consists of two layers that are continuous with each other: the parietal peritoneum and the visceral peritoneum. Both types are made up of simple squamous epithelial cells called mesothelium. The parietal peritoneum lines the internal surface of the abdominopelvic wall. The visceral peritoneum invaginates to cover the majority of the abdominal viscera.

The term “peritoneal cavity”, as used herein, refers to the potential space between the parietal and visceral peritoneum. It normally contains only a thin film of peritoneal fluid, which consists of water, electrolytes, leukocytes and antibodies. This fluid acts as a lubricant, enabling free movement of the abdominal viscera, and the antibodies in the fluid fight infection. While the peritoneal cavity is ordinarily filled with only a thin film of fluid, it is referred to as a potential space because excess fluid can accumulate in it.

In peritoneal dialysis, a soft plastic tube (catheter) is placed in the peritoneal cavity by surgery to enable the insertion of sterile peritoneal dialysis fluids. The catheter is made of a soft, flexible material (usually silicone) and has cuffs (which are like Velcro), which are placed under the skin. Skin tissue grows into them to hold the catheter in place. The end of the catheter inside the abdomen has multiple holes to allow fluid to flow in and out. After the filtering process is finished, the fluid leaves the body through this catheter.

There are two kinds of peritoneal dialysis: continuous ambulatory peritoneal dialysis (CAPD), and automated peritoneal dialysis (APD). The basic treatment is the same for each. However, the number of treatments and the way the treatments are done make each method different. The CAPD is “continuous,” machine-free and done while the patient goes about his/her normal activities such as work or school. Treatment is done by placing about two quarts of PDF into the abdomen (peritoneal cavity) by hooking up a plastic bag of PDF to the catheter, and later draining it. Raising the plastic bag to shoulder level causes gravity to pull the fluid into the body. When empty, the plastic bag is removed and thrown away. The dialysate in the peritoneal cavity absorbs wastes removed from the blood and is drained from the abdomen and thrown away after a few hours. When the PDF is fresh, it absorbs wastes quickly. As time passes, filtering slows. For this reason, the patient needs to repeat the process of emptying the used dialysate and refilling the abdomen with fresh PDF, a process termed “exchange”, usually three, four, five or six times in a 24-hour period, mostly while the patient is awake during normal activities. Each exchange takes about 30 to 40 minutes. Some patients prefer to do their exchanges at mealtimes and at bedtime.

Automated peritoneal dialysis differs from CAPD in that a machine (cycler) delivers and then drains the cleansing fluid for the patient. The treatment usually is done at night while sleeping.

The type of peritoneal dialysis that is best for a certain patient depends on the patient's personal choice and medical condition as well as the physician discretion.

With peritoneal dialysis, the patient can control extra fluid more easily, and this may reduce stress on the heart and blood vessels. The patient is able to eat more and use fewer medications, can do more of daily activities and it is easier to work or travel. Peritoneal dialysis is an effective form of dialysis and has been proven to be as good as hemodialysis.

In some embodiments, the peritoneal dialysis in a contemplated method is CAPD. Additionally or alternatively the peritoneal dialysis is APD.

In some embodiments, a contemplated method results in reduction in blood glucose levels during PD as compared to PD without introducing a SGLT inhibitor.

In some embodiments, a disclosed method results in reduced peritoneal membrane damage.

An effective amount or a therapeutically effective amount of a compound e.g., a SGLT inhibitor, or composition e.g., a PDF (herein referred to as APIs), and/or a formulation thereof, is a quantity of API and/or formulation sufficient to achieve a desired effect in a subject being treated. An effective amount of an API can be administered in a single dose, or in several doses, for example daily, during a course of treatment. However, the effective amount of the API will be dependent on the API applied, the subject being treated, the severity and type of the affliction, and the manner of administration of the compound.

“Administration” as referred to herein is introduction of the API or a or formulation comprising it as defined herein into a subject by a chosen route. Administration of the active compound or pharmaceutical composition can be by any route known to one of skill in the art, and as appropriate for the particular condition and location under treatment. Administration can be local or systemic.

Examples of local administration include, but are not limited to, topical administration, subcutaneous administration, intramuscular administration, intrathecal administration, intrapericardial administration, intra-ocular administration, topical ophthalmic administration, intraperitoneal administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration, subcutaneous administration, intraperitoneal administration, intraduodenally administration, and intravenous administration when such administration is targeted to the vasculature supplying a particular organ. Local administration also includes the incorporation of the API and/or formulation comprising it into implantable devices or constructs, such as vascular stents, infusion pumps or other reservoirs, which release the API over extended time intervals for sustained treatment effects.

Systemic administration includes any route of administration designed to distribute the API or a formulation comprising it widely throughout the body via the circulatory system. Thus, systemic administration includes, but is not limited to, intra-arterial and intravenous administration, topical administration, subcutaneous administration, intraduodenally administration, intramuscular administration, or administration by inhalation, when such administration is directed at absorption and distribution throughout the body by the circulatory system.

In some exemplary embodiments described herein, a PDF is administered intraperitoneally in the course of a peritoneal dialysis as described herein.

The one or more SGLT inhibitors may be administered or introduced to a subject in need thereof together with the PDF, in a single administration, e.g., intraperitoneally. In some embodiments, a single administration is provided by the use of a PDF containing one or more SGLT inhibitors, or a formulation comprising same, as single unit dosage form. In exemplary embodiments, phlorizin, an analog thereof and/or a derivative thereof is the SGLT inhibitor, and a PDF containing same is a phlorizin peritoneal dialysis fluid (PPDF), administered as a single unit dosage from.

In some embodiments, PPDF administered to a subject inflicted with renal failure is more effective in preventing blood glucose elevation during PD as compared to PDF and the SGLT inhibitor being administered separately, as disclosed, for example in Examples 4 and 5 herein.

In some embodiments, a single administration is provided by the use of a pharmaceutical composition or a formulation comprising a PDF and one or more SGLT inhibitors as two or more dosage unit forms administered together in a single administration.

In some embodiments, a PDF and at least one SGLT inhibitor are introduced to the patient separately, e.g., in separate administrations or via a co-administration. The at least one SGLT inhibitor can be administered via any suitable route. For example, the administration route may be intraperitoneal, subcutaneous, intramuscular, intradermal, transdermal, intranasal and/or oral.

In exemplary embodiments, the separate administration of at least one SGLT inhibitor is intraperitoneal, i.e., to the peritoneal cavity.

In further exemplary embodiments, the SGLT inhibitor is administered subcutaneously.

In some embodiments, one or more SGLT inhibitors are contained or provided in the PDF and administered together with the PDF as a single unit dose form, and additional dose(s) of same or different SGLT inhibitor(s) is/are administered separately.

In some embodiments, a SGLT inhibitor being provided or administered in accordance with a contemplated method is a SGLT1 inhibitor, for example, but not limited to, a SGLT inhibitor which is selective to SGLT1. In some embodiments, the SGLT inhibitor is SGLT5, for example, a SGLT inhibitor which is selective to SGLT5. In some embodiments, the SGLT inhibitor is a dual SGLT1 and SGLT5 inhibitor. In some embodiments, the SGLT inhibitor is a dual SGLT1/SGLT2 inhibitor. In some embodiments, the SGLT inhibitor is capable of inhibiting SGLT2 and at least one of SGLT1 and SGLT5.

In exemplary embodiments, at least one SGLT inhibitor administered to a subject in the course of a PD or a of contemplated method described herein, wherein the at least one SGLT inhibitor is, for example, phlorizin a phlorizin analog and/or a phlorizin derivative. In certain embodiments, the at least one SGLT inhibitor is selected from a phlorizin derivative capable of inhibiting SGLT1. For example, the phlorizin derivative may an O-glucoside analog such as T-1095, remogliflozin and sergliflozin, or a C-glucoside analog such as, but not limited to, apagliflozin, canagliflozin, and empagliflozin.

In exemplary embodiments, the at least one SGLT inhibitor is a phlorizin inhibitor capable of inhibiting SGLT5.

In exemplary embodiment the dual SGTL1/SGLT2 inhibitor is Sotagliflozin.

Embodiments described herein, in context of a disclosed method, provide the administration to a subject in need thereof of at least two different SGLT inhibitors in the course of a PD. In exemplary embodiments, the subject undergoing PD is provided with a PDF containing at least a SGLT1 inhibitor and a SGLT5 inhibitor.

In accordance with a contemplated method described herein, the SGLT inhibitor is introduced at a concentration of form about 0.1 to about 50 μM. For example, from about 0.2 to about 20 μM, from about 0.4 to about 10 μM or about 0.6 μM.

Treating a disease, as referred to herein, means ameliorating, inhibiting the progression of, delaying worsening of, and even completely preventing the development of a disease, for example ameliorating or delaying worsening of the clinical condition in a person who has a renal failure. Treatment refers to a therapeutic intervention that ameliorates a sign or symptom of a disease or a pathological condition after it has begun to develop. In particular examples, however, treatment is similar to prevention, except that instead of complete inhibition, the development, progression or relapse of the disease is inhibited or slowed.

### Kits

In an aspect of the present disclosure, there is provided a kit comprising a PDF and one or more SGLT inhibitors, or a formulation comprising it, as defined in any of the embodiments described herein and, optionally, instructions and means for administration of the active agents and/or the formulation to a subject in need thereof.

In some embodiments a disclosed kit is a peritoneal dialysis kit.

A contemplated kit is useful for treatment of a renal failure disease, disorder or condition as defined herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

As used herein the term “about” refers to ±10%.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, chemical, biochemical, microbiological and/or recombinant DNA techniques. Such techniques are thoroughly explained in the literature. See for example, Guide to Research Techniques in Neuroscience (Second Edition), Matt 2015; Elsevier's Integrated Review Biochemistry (Second Edition), 2012. Other general references are provided throughout this document. The procedures therein are believed to be well known in the art and are provided for the convenience of the reader. All the information contained therein is incorporated herein by reference.

### Materials and Methods

Phlorizin extracted from apple wood was purchased from Sigma-Aldrich®, Israel.

Peritoneal dialysis fluid used was Dianeal® purchased from Baxter Healthcare S.A, Ireland.

### In Vivo Assays

### Bilateral Ureteral Obstruction

Renal failure in mice and rats was induced by bilateral ureteral ligation, based on the procedure described, for example, in Kim et al. (2015) (Am J Physiol Renal Physio., 308(2):F131-139) and references cited therein. Briefly, mice were anesthetized with an intraperitoneal injection of a cocktail containing ketamine (200 mg/kg body weight) and xylazine (16 mg/kg body weight). After exposure of both the left and right kidneys through flank incision, right and left ureters were ligated completely near the kidney pelvis using a 5-0 silk tie.

### In Vitro Assays

### (i) Reverse Transcription Polymerase Chain Reaction (RT-PCR)

Reverse transcription polymerase chain reaction (RT-PCR) is the technology to study gene expression based on mRNA detection and quantitation, by which mRNA molecules are converted into their complementary single stranded DNA (cDNA) sequences by the enzyme reverse transcriptase (RT), followed by the amplification of the newly synthesized cDNA by standard polymerase chain reaction (PCR) procedures. DNA polymerase is used to convert the single-stranded cDNA into double-stranded DNA in the PCR process. These cDNA molecules are then used as templates for a PCR reaction. The value of RT-PCR is that it can be used to determine if an mRNA species is present in a sample or to clone a cDNA sequence. RT-PCR is a two-step process. It involves reverse transcription of purified RNA by RT via an appropriate method for priming, and then amplification of first-strand cDNA using some variant of PCR. Both steps can be readily executed using relevant commercially available kits.

Briefly, a RT-PCR process was conducted as follows:

(i) after RNA was released from the visceral peritoneum, kidney and small intestine of mice cellular material through extraction, an aliquot of the extracted sample was added to a reaction mixture which contained reverse transcriptase enzyme, primers specific for the target of interest (SGLT5 gene), and nucleotides;

(ii) if the target was present, primers annealed to the RNA strand;

(iii) reverse transcriptase enzyme synthesized a complementary DNA strand, extending from the primer;

(iv) the temperature was raised to 95° C., and the RNA/DNA strands were denatured;

(v) the temperature was lowered, allowing primers to anneal to the newly formed cDNA;

(vi) the polymerase enzyme synthesized a new DNA strand, extending from the primer; and

(vii) multiple cycles geometrically increased the number of copies of DNA.

RT-PCR may be performed as one- or two-step procedure. In some embodiments, a one-step procedure was employed, wherein reverse transcription of mRNA was performed in the same reaction tube as the polymerase chain reaction. In alternative embodiments, a two-step procedure was employed, wherein transcription of the RNA to cDNA was performed first as described above. Transcription occurred between 40° C. and 50° C., depending on the properties of the reverse transcriptase enzyme utilized; products of that reaction were then amplified in a separate reaction.

Primers used were sequence specific primers, namely, custom made primers that specifically targeted SGLT12 and SGLT5 mRNAs. These primers were purchased from Sigma-Aldrich®, Israel.

In some embodiments, expression of SGLT1 and SGLT5 gene expression was measured by RT-PCR performed as end-point RT-PCR. Expression of SGLT1 and SGLT5 was not quantified. The presence of SGLT1 and SGLT15 was confirmed by the presence and expected size of the PCR products. In the case of SGLT5, which was not described in the peritoneum before, the PCR product was sequenced and aligned with the published sequence of mouse SGLT5. SGLT5 DNA was sequenced by the Ben-Gurion University “core laboratory” on ABI PRISM® 3100 Genetic Analyzer (Applied Biosystems).

### Example 1

### Identification of SGLT Isoforms in Peritoneal Membranes

To identify the SGLT isoforms in the peritoneal membrane, reverse transcription polymerase chain reaction (RT-PCR) analysis was performed as described in Materials and Metods. mRNA was extracted from the visceral peritoneum, kidney and small intestine of CD-1® mice using a GENEzol™ TriRNA pure kit RNA Tissue Kit (Generaid™, New Taipei, Taiwan), according to manufacturer protocol. cDNA was synthesized using a cDNA reverse transcription kit (Applied Biosystems®; Foster City, Calif.). PCR assays were done using a DreamTaq™ DNA Polymerase (Thermo Scientific™, Waltham, Mass.).

The primers used were:

SGLT1 FW
CTGCTAGCAATCACTGCCCT;
SGLT1 RE
CTGCTAGCAATCACTGCCCT;
SGLT5 FW
ATGGCCAAACACCCCAGAAA;
and
SGLT5 RE
TTTCTCCCCATATCTGACTTTGT.

These oligonuclotides were designed using, for example, Primer-Blast™ (https://www.ncbi.nlm.nih.gov/tools/primer-blast/) and purchased from Sigma-Aldrich®, Israel.

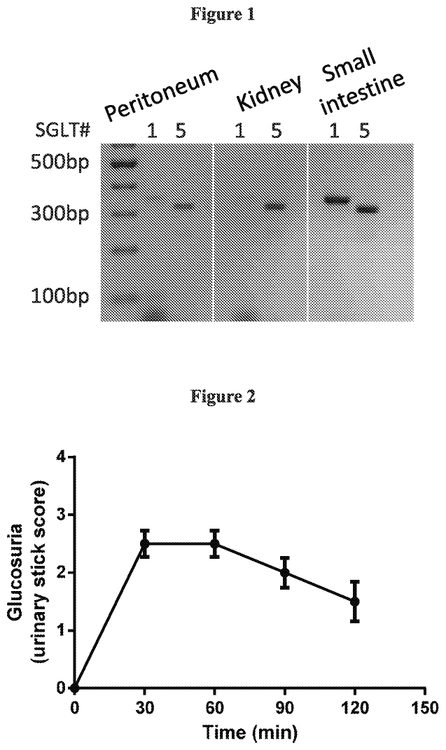

FIG. 1 demonstrates the presence of SGLT1 (352 bp) and SGLT5 (322 bp) isoforms in the peritoneum. SGLT1 and SGLT5 were also characterized in the kidney and the small intestine of CD-1® mice, which actually were used as positive controls as they are known to express the same isoforms (the small intestine express elevated levels of SGLT1 and SGLT5). Product of the RT-PCR process were run on a 2% agarose gel stained with ethidium bromide.

In addition to the RT-PCR analysis, DNA sequencing was done to the SGLT5 band to confirm the presence of SGLT5 in the peritoneum.

### Example 2

### Effect of SGLT Inhibition on Glucose Reabsorption by Kidneys and Peritoneum

In the kidneys, reabsorption of glucose occurs mainly in the proximal tubule during the formation of primary urine and is mediated by two different transport proteins, SGLT1 and SGLT2. To expend the knowledge and understanding of SGLTs involvement in glucose absorption during PD treatment, an inhibition of glucose absorption by phlorizin (SGLT1/SGLT2 inhibitor) and dapagliflozin (specific SGLT2 inhibitor) just before PD treatment was performed.

Fifteen minutes before peritoneal fluid injection, phlorizin (20 mg/kg) was administered to healthy CD-1® mice (n=6) by a single subcutaneous injection. Dapagliflozin (0.1 mg/kg) was given to the mice by oral gavage. Control mice were not given any of the SGLT blockers. Tail blood glucose was tested by glucose test meter immediately before (time 0) and during 2 hours after injection. Glucose levels in the urine were tested by standard urine sticks (Multistix® 10 SG, Siemens Healthcare Diagnostics, Tarrytown, N.Y.).

As seen in FIG. 2, glucose levels in the mice urine were elevated as a result of phlorizin injection. Phlorizin caused significant glycosuria which persisted for more than 2 hours. A similar effect was observed for dapagliflozin (results not shown), as an evidence of effectiveness of the SGLT2 inhibitor on kidney glucose reabsorption in mice. Glucose levels in mice blood that increased following intraperitoneal glucose-containing PD fluid exposure in the control group were significantly lower after administration of these inhibitors (results not shown), which may be attributed to decreased peritoneal glucose transport. These results indicate that peritoneal SGLT transporters are probably involved in glucose transport during PD treatment and their inhibition may help to prevent excessive glucose absorption and negative metabolic and toxic consequences that are related to it.

### Example 3

### Effect of SGLT Inhibition on Glucose Transport

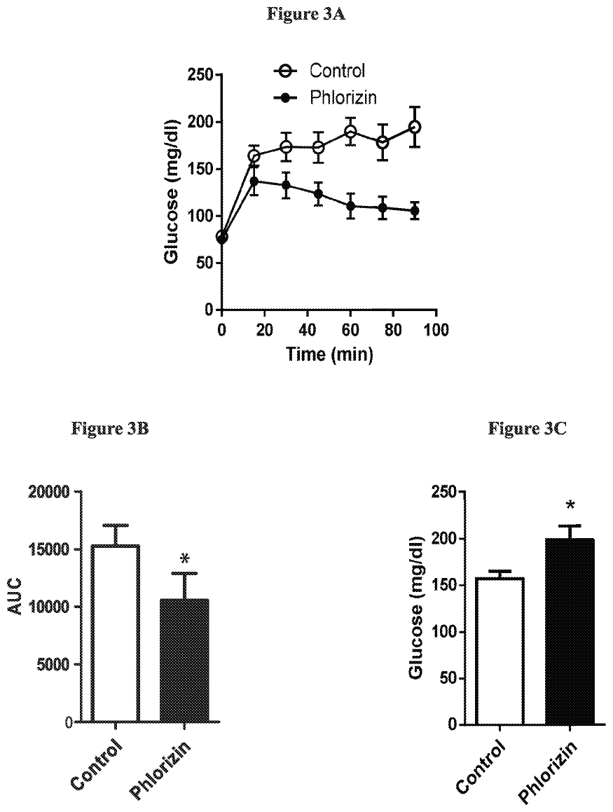

To explore the effect of phlorizin on peritoneal glucose transport, a renal failure mice model was induced by a known bilateral ureteric ligation method as described in Materials and Methods. Twenty four hours after the renal failure induction, CD-1® mice (n=5) were subcutaneously (SC) injected with a single dose of either SGLT inhibitor phlorizin (20 mg/kg) or saline as a control Immediately after subcutaneous administration, mice peritoneum was injected with 2 ml dialysis fluid. A standard commercial PD fluid (Dianeal® 1.5% dextrose), diluted 1:4 in saline (comprising 0.375% glucose) was used. Blood glucose levels were measured by glucometer at indicated time points. Area under the curve (AUC) was calculated for all mice and used to compare the two groups. Results are shown in FIGS. 3A and 3B; P<0.05. Concentrations of glucose in the peritoneum fluid measured 30 min after administration of PDF are depicted in FIG. 3C.

Compared to control mice treated with PDF alone, in the peritoneum fluid of phlorizin-treated group, glucose levels measured at 30 minutes from instillation of PDF were significantly elevated (FIG. 3C).

As shown in FIGS. 3A and 3B, the nonselective SGLT inhibitor phlorizin mitigated hyperglycemia following peritoneal exposure to glucose containing PDF while maintaining elevated glucose levels in the peritoneum (FIG. 3C).

Dependency of glucose transport on SGLT inhibition during peritoneal dialysis with PDF not containing sodium was assessed. CD-1® mice (n=5) with a renal failure (induced, for example, as described above), were injected SC with phlorizin (20 mg/kg) or saline (control) and then injected with sodium free PDF (2 ml), containing 0.375% glucose to the peritoneum.

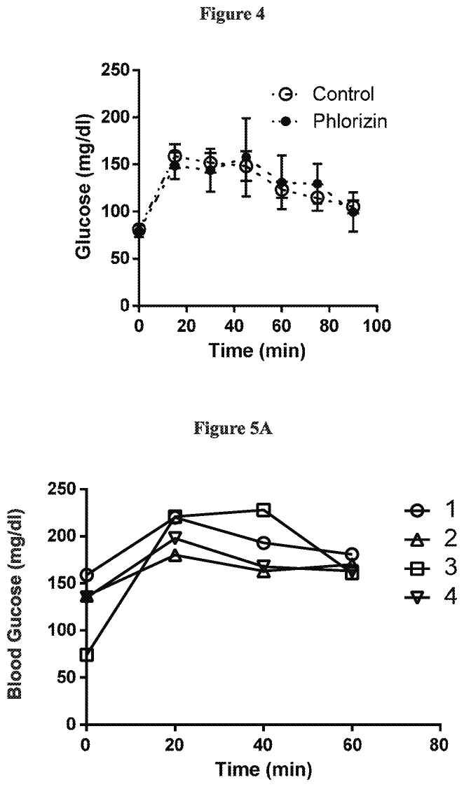

As seen in FIG. 4, no differences in blood glucose levels were identified between phlorizin treated mice and control group, indicating inactive cooperative sodium-glucose transport by SGLT in absence of sodium. It is to be noted that sodium-free PDF cannot be used clinically because it will cause dangerous disturbances in blood sodium level. All currently known PDFs contain sodium at concentration close to normal blood level. During peritoneal dialysis, equilibration of electrolytes concentrations in blood and dialysate occurs.

### Example 4

### Effect of Phlorizin Peritoneal Dialysis Fluid (PPDF) in Mice

A further animal study was designed to explore the phlorizin effect on peritoneal glucose transport, following administration of phlorizin peritoneal dialysis fluid (PPDF) (as opposed to separate SC administration of SGLT inhibitor and DF).

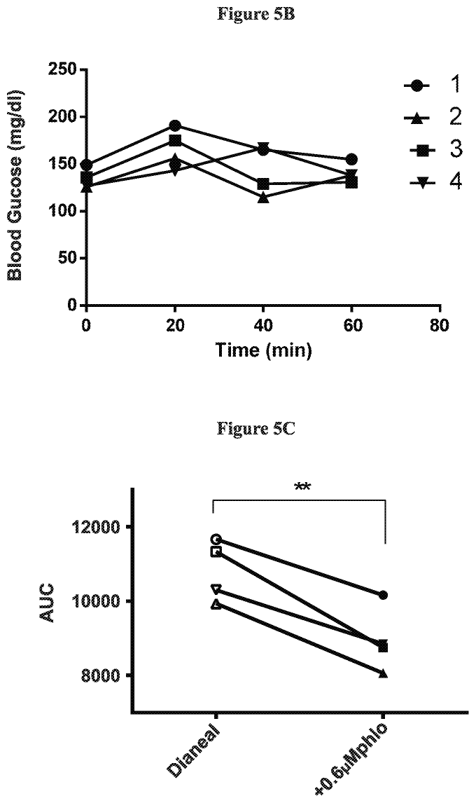

CD-1® uremic mice (n=4) were injected intraperitoneally with 2 ml dialysis fluid (Dianeal® 1.5% dextrose) mixed with phlorizin (phlorizin-containing peritoneal dialysis fluid; 0.6 μM phlorizin) or without phlorizin (control group, phlorizin-free PDF). Blood glucose levels were measured by glucometer at indicated time points, and area under the curve (AUC) was calculated for all mice and used to compare the two groups (FIG. 5C; P<0.01).

FIGS. 5A and 5B depict blood glucose levels, and FIG. 5C compares AUC obtained for mice treated with Dianeal® PDF and mice treated with Dianeal® supplemented with 0.6 μM phlorizin (PPDF). As seen in the figures, the addition of phlorizin directly to the peritoneal dialysis fluid was significantly more effective in reducing blood glucose levels in CD-1® mice as compared to separate applications of phlorizin and PDF.

### Example 5

### Phlorizin Effect of on Glucose Absorption from Peritoneal Fluid in Rats

The effect of phlorizin on glucose absorption during peritoneal dialysis was further assessed using renal failure models in animals lager than mice such as rats and rabbits. Comparing to mice, the advantage of rats and rabbits is the possibility to perform dialysis with a larger volume of peritoneal dialysis fluid which enables multiple sampling of the dialysate in real time. Rabbits allow dialysis to be performed in a way very similar to that used in human patients. A study with rats is exemplified herein. In this experiment, rats were treated with PDF with phlorizin (PPDF) or without phlorizin. Glucose concentrations in blood and dialysate were monitored, and the dilution of PDF was measured using high molecular weight fluorescent tracer (FITC-Dextran) that does not diffuse from the peritoneum. A corresponding study with rabbits may be performed in a rabbit model for peritoneal dialysis based, for example, on the protocol described in Garosi and Paolo (2001) (Nephrol Dial Transplant. 16(3):664-665), and the protocol described herein for rats.

### Peritoneal Dialysis Model

Animal experiments were approved by local Animal Care Committee. Male Sprague Dawley (SD) rats 200-250 gr, we purchased from Envigo RMS (Jerusalem, Israel), allowed to acclimatize for 1 week before the beginning of the experiment. Animals were anesthetized by intramuscular administration of a mixture of ketamine (200 mg/kg body wt) and xylazine (16 mg/kg body wt). Renal failure was induced by bilateral ureteral ligation as described in Materials and Methods. Twenty four hours after ureteral ligation, fifteen uremic rats were randomly divided into two groups: control group (n=7); and phlorizin group (n=8). After anesthesia temporary catheters were introduced into the peritoneal cavity.

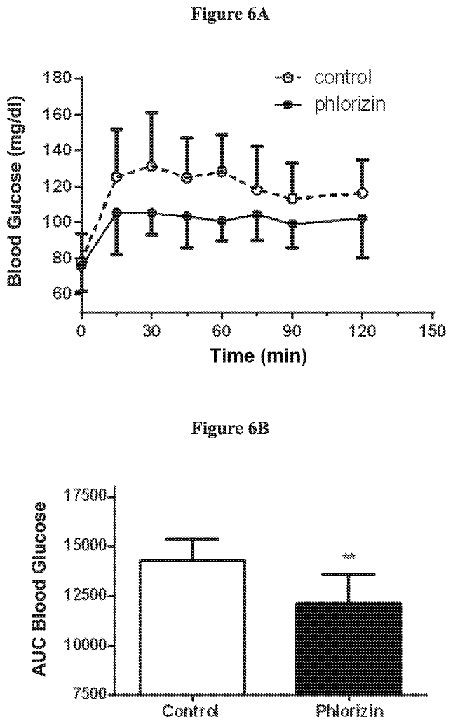

The catheters were used to infuse 20 ml of glucose containing PDF (1.5% Dianeal® diluted 1:4 with 0.9% normal saline) with or without phlorizin (100 μg/ml). To PDF of both group 100 μl of fluorescein isothiocyanate-dextran was added, average molecular weight 2,000,000 (FITC Dextran −2000, 100 μg/ml, Sigma-Aldrich®, Israel). Dialysate and tail blood samples were obtained during 120 min dwell, every 15 min. Blood glucose level was measured by standard glucometer, dialysate glucose was measured in the chemistry laboratory of the Soroka Medical Center. Dilution of FITC-dextran was detected by florescence of dialysis fluid samples measured with a 96-well fluorimeter (SpectraMax® Paradigm® plate reader, Molecular Devices) at an emission wavelength of 535 nm and an excitation wavelength of 485 nm. Concentrations of unknown samples were calculated from a standard curve by extrapolation in a linear regression model. Results are schematically presented in FIGS. 6-8.

Significantly lower blood glucose levels in phlorizin as compared to control group as shown in FIGS. 6A and 6B, clearly indicate that phlorizin added to PDF was able to diminish blood glucose load during intraperitoneal PDF exposure. This effect of the SGLT inhibitor may help decreasing multiple metabolic complications of PD treatment caused by glucose toxicity.

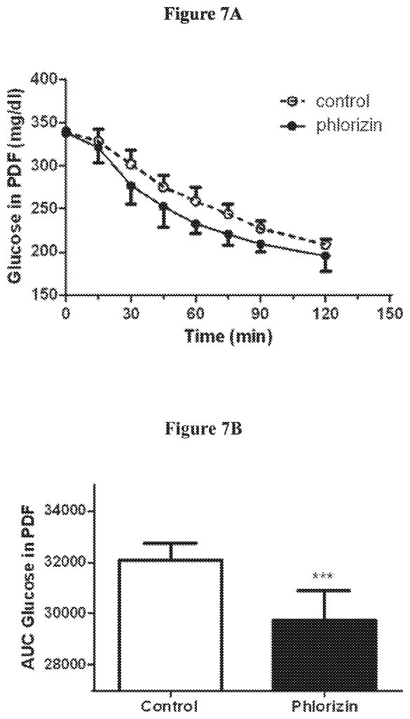

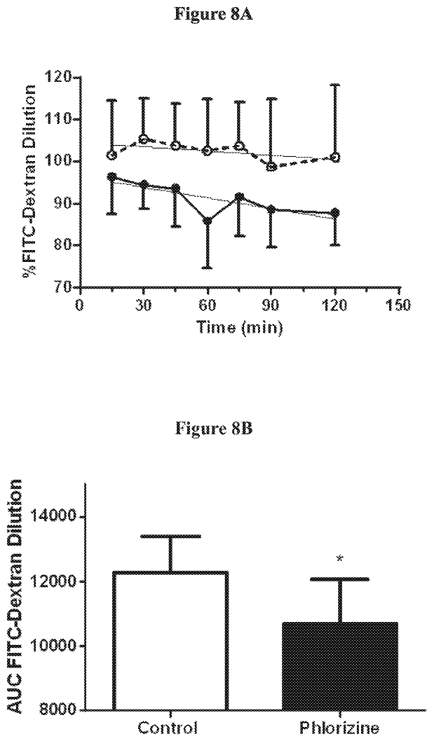

Looking at PDF glucose level as shown in FIGS. 7A-7B, it is seen that glucose significant declined in the phlorizin group compared to control group (p<0.001). Also, as seen in FIGS. 8A-8B, FITC-dextran was significant diluted in the phlorizin group as compared to control (P<0.05). The results as shown in FIGS. 7 and 8 imply that the decrease in dialysate glucose concentration is caused by dilution. The significantly higher dilution of FITC-dextran in animals treated with phlorizin-containing PDF compared to phlorizin-free PDF is a direct evidence of increased ultrafiltration, and further supports the use of SGLT inhibitors such as phlorizin directly introduced into a PDF (i.e., forming a single unit dosage form) in order to improve performance of PDF even with low glucose concentrations.

### Example 6

### Production and Characterization of Phlorizin Peritoneal Dialysis Fluid (PPDF)

A SGLT inhibitor that will be given directly in the peritoneal dialysis fluid, where the inhibitor is designed to lower blood glucose concentration by preventing glucose reabsorption by specifically targeting SGLTs, presents an advanced approach in peritoneal dialysis and is highly desired. Such new SGLTs inhibitors that will be safe to use in the treatment of ESKD patients, may provide reduced glucose load during PD, thereby improve patients' outcome and lessen peritoneal membrane damage.

A screening of PPDF solutions containing 0.2-20 μM phlorizin are scored to establish the concentrations needed to obtain the desired pharmacological effects. The PPDF comprises standard glucose-based PDF (Dianeal® 1.5% dextrose). Parameters such as pH, viscosity, storage stability, etc. are measured. HPLC is used to determine the content of phlorizin in the analyzed samples and to measure the stability of the phlorizin.

The following parameters are tested and considered in the screening process:

a. inhibition of glucose uptake from the peritoneal fluid to the blood;

b. glycosuria assay: the potential accumulation of phlorizin in the blood is tested;

c. inhibition of glucose uptake in the gut (a potential side effect of phlorizin, IC50=0.4 μM). Blood levels are measured following oral administration of glucose; and

d. physiological and behavior parameters of treated animals.

### INCORPORATION BY REFERENCE

The entire contents of all patents, published patent applications, websites, and other references cited herein are hereby expressly incorporated herein in their entireties by reference.

1. A peritoneal dialysis fluid (PDF) comprising at least one inhibitor of a sodium-glucose co-transporter (SGLT inhibitor).

2. A pharmaceutical composition comprising a dialysis fluid, at least one inhibitor of a sodium-glucose co-transporter (SGLT inhibitor), and a pharmaceutically acceptable excipient.

3. (canceled)

4. The pharmaceutical composition of claim 2, wherein the dialysis fluid is a peritoneal dialysis fluid (PDF).

5. The pharmaceutical composition of claim 4, wherein the PDF and the at least one SGLT inhibitor form at least one of: a single unit dosage form, or two or more unit dosage forms.

6. (canceled)

7. The PDF of claim 1, wherein the SGLT inhibitor is at least one of a SGLT1 inhibitor, SGLT5 inhibitor, or a dual SGLT1/SGLT5 inhibitor.

8. The pharmaceutical composition of claim 2, wherein the SGLT inhibitor is at least one of a SGLT1 inhibitor, a SGLT5 inhibitor, or a dual SGLT1/SGLT5 inhibitor.

9-10. (canceled)

11. The PDF of claim 1, wherein the SGLT inhibitor is at least one of phlorizin, a phlorizin analog, a phlorizin derivative.

12. The PDF of claim 11, wherein the SGLT inhibitor is a phlorizin derivative or a phlorizin analog characterized as being capable of at least one of:
(a) inhibiting SGLT1;
(b) being selective to SGLT1;
(c) inhibiting SGLT1 and SGLT2; or
(d) being selective to SGLT5.

13-15. (canceled)

16. The PDF of claim 12, wherein the SGLT inhibitor is a phlorizin analog selected from the group consisting of O-glucoside analogs and C-glucoside analogs.

17. The PDF of claim 1, comprising at least two SGLT inhibitors.

18. The PDF of claim 17, wherein the at least two SGLT inhibitors comprise a SGLT1 inhibitor and a SGLT5 inhibitor.

19. The PDF of claim 1, comprising from about 0.1 to about 50 μM, or from about 0.2 to about 20 μM of at least one SLGT inhibitor.

20-23. (canceled)

24. A method of treating a subject in need of a peritoneal dialysis, comprising the steps of:
administering an effective amount of a peritoneal dialysis fluid (PDF) into the peritoneal cavity of the subject; and
administrating an effective amount of at least one inhibitor of a sodium-glucose co-transporter (SGLT inhibitor) to the subject, thereby treating the subject in need of a peritoneal dialysis.

25. The method of claim 24, wherein the at least one SLGT inhibitor is administered together with the PDF into the peritoneal cavity.

26. (canceled)

27. The method of claim 24, wherein the at least one SLGT inhibitor is administered orally or subcutaneously, and the PDF is administered intraperitoneally.

28. The method of claim 24, wherein the SGLT inhibitor is at least one of a SGLT1 inhibitor, a SGLT5 inhibitor, or a dual SGLT1/SGLT5 inhibitor.

29. The method of claim 24, affording, to a subject undergoing peritoneal dialysis, at least one of: reduction in peritoneal membrane damage, or reduction of blood glucose levels.

30-31. (canceled)

32. The method of claim 24, for treating a renal failure disease, disorder or condition in the subject.

33. The pharmaceutical composition of claim 2, wherein the SGLT inhibitor is at least one of phlorizin, a phlorizin analog, or a phlorizin derivative, and is characterized as being at least one of:
(a) capable of inhibiting SGLT1;
(b) being selective to SGLT1;
(c) capable of inhibiting SGLT1 and SGLT2;
(d) being selective to SGLT5; or
(e) being a phlorizin analog selected from the group consisting of O-glucoside analogs and C-glucoside analogs.

34. The pharmaceutical composition of claim 2, comprising at least two SGLT inhibitors being a SGLT1 inhibitor and a SGLT5 inhibitor.


SEQUENCE LISTING (4 sequences)

SEQ ID NO: 1 - DNA, 20 bp, Artificial Sequence
  Synthetic
  misc_feature: SGLT1 FW
ctgctagcaatcactgccct

SEQ ID NO: 2 - DNA, 20 bp, Artificial Sequence
  Synthetic
  misc_feature: SGLT1 RE
ctgctagcaatcactgccct

SEQ ID NO: 3 - DNA, 20 bp, Artificial Sequence
  Synthetic
  misc_feature: SGLT5 FW
atggccaaacaccccagaaa

SEQ ID NO: 4 - DNA, 23 bp, Artificial Sequence
  Synthetic
  misc_feature: SGLT5 RE
tttctccccatatctgactttgt